(19) United States
(12) Patent Application Publication
(10) Pub. No.: US 2026/0240584 A1
(43) Pub. Date: Aug. 20, 2026

(54) A BONE NAIL MOUNTING HOLE POSITIONING DEVICE

(71) Applicant: KAOHSIUNG MEDICAL UNIVERSITY, Kaohsiung City (TW)

(72) Inventors: Yin-Chih Fu, Kaohsiung City (TW); Yu-Chiao Sue, Kaohsiung City (TW); Tien-Ching Lee, Kaohsiung City (TW); Yan-Hsiung Wang, Kaohsiung City (TW); Mei-Ling Ho, Kaohsiung City (TW); Chung-Hwan Chen, Kaohsiung City (TW); Li-Ting Kao, Kaohsiung City (TW)

(73) Assignee: KAOHSIUNG MEDICAL UNIVERSITY, Kaohsiung City, TW (TW)

(21) Appl. No.: 19/159,325

(22) PCT Filed: Feb. 29, 2024
(86) PCT No.: PCT/CN2024/079174

§ 371 Date: Aug. 25, 2025

Related U.S. Application Data
(60) Provisional application No. 63/490,002, filed on Mar. 14, 2023.

(51) Int. Cl. A61B 17/90

(52) U.S. Cl. CPC A61B 17/90

(57) ABSTRACT

The invention is related to a bone nail mounting hole positioning device, comprising: a bone nail, including a screw hole to be locked with a screw, for implanting into a body or a bone of a subject to be locked with the screw, in which the screw hole is penetrating into a wall of the bone nail; a laser source for emitting a laser light of an incident light signal; an optical fiber, including a head portion, a light source optical fiber connected with the laser source for guiding the laser light to be incident on the bone nail of the subject, and a receiving optical fiber for receiving a reflection light signal reflected from the bone nail of the subject, in which the head portion of the optical fiber is movably contacting the body or the bone of the subject implanted with the bone nail; and a receiving device, connected with the receiving optical fiber, including a detector and a power meter, so as to defect, measure and indicate an energy difference factor, in which the smaller the energy difference factor, the closer the approaching of the screw hole to the screw nail to be locked, wherein the energy difference factor is a power ratio between the reflection light signal and the incident light signal.

This application claims the benefit of PCT Patent Application No. PCT/CN2024/079174 field on Feb. 29, 2024, claiming a priority date of Mar. 14, 2023, which is incorporated by reference herein in its entirety. Meanwhile, this application is preceded by a corresponding US Provision Patent Application No. 63/490,002 filed on Mar. 14, 2023.

### FIELD OF THE INVENTION

The present invention relates to a bone nail mounting hole positioning device, especially to a reflective bone nail mounting hole positioning device, that is, by detecting an laser light energy factor reflected from a different locations of the bone nail or bone plate, a correct bone nail mounting hole is positioned position

### BACKGROUND OF THE INVENTION

Lower limb fractures are one of the most commonly treated fractures in orthopedic clinics, and are mainly fixed by using intramedullary steel nails and minimally invasive interlocking bone plates. However, at present, the process of locating the fixing holes (screw holes) of the intramedullary nails and locking the screws is difficult and time-consuming, and it is easy to cause secondary injuries. The most commonly used traditional freehand positioning uses fluoroscopy X-ray machines to find a perfect round hole, which takes a long time to operate, causes a lot of radiation exposure, and relies more on the experience of the surgeon. Although many modified positioning methods have been proposed, such as extended drilling guidance, computer navigation systems, light transmission methods, remote positioning wires, and other freehand positioning techniques, and even positioning methods using two overlapping bone nails, most of these methods are expensive, large in size, or providing a steep learning curve for physicians.

### SUMMARY OF THE INVENTION

The present invention aims to provide a reflective bone nail mounting hole positioning device that can perform the positioning of bone nail screw holes (through holes) in a correct, fast, convenient and inexpensive manner. The design principle mainly uses the characteristics that bone nails (bone marrow nails) and bone plates may reflect signals, to design a laser positioning system of the instrument. As there is no correct positioning, the laser incident light signal will be reflected by the bone nail or bone plate, and the receiving device will receive the reflection signal, and as there is correct positioning, the laser incident light signal will not be reflected because it will penetrate the screw hole and will not be reflected, such that the reflected light signal received by the receiving device will be greatly reduced, whereby a correct positioning of the screw hole can be judged via detecting the difference in the energy of the reflected light signal, that is, the larger energy difference between the incident light signal and the reflected light signal, the smaller the energy difference factor (a power ratio between the reflected light signal and the incident light signal), indicating the closer to the screw hole, that is, the more accurate the positioning.

A bone nail screw hole positioning device according to the present invention comprises: a bone nail with a screw hole to be locked by a screw, for being implanted into a body or bone of a subject to be locked, in which the screw hole runs through a wall of the bone nail; a laser source for emitting a laser light with an incident light signal; an optical fiber tube, including a head portion, a light source optical fiber connected to the laser source for guiding the laser light to be incident on the bone nail of the subject, and a receiving optical fiber for receiving a reflected light signal reflected from the bone nail implanted into the body or bone of the subject, wherein the head portion of the optical fiber tube is movably contacting the body or bone of the subject implanted with the bone nail; and a receiving device, including a detector connected to the receiving optical fiber, and a power meter connected to the detector, wherein the incident light signal emitted by the laser source is transmitted through the light source optical fiber to the head portion of the optical fiber tube, and the receiving optical fiber receives the reflected light signal reflected respectively from a different location of the body or bone of the subject, so that the reflected light signal is transmitted to the power meter for measuring via detection of the detector, so as to obtain and indicate an energy difference factor, in which the energy difference factor is a power ratio between the received reflected light signal and the incident light signal, and the smaller the energy difference factor, the closer to the screw hole to be locked, in which the incident light signal is correctly incident on the screw hole and passes therethrough, such that no reflected energy is generated and thus detected by the detector of the receiving device, resulting in a larger difference between a received light energy and an emitted light energy to obtain a smaller energy difference factor indication, whereby a correct position of the screw hole is positioned via the measurement and indication of the power meter.

### BRIEF DESCRIPTION OF THE DRAWING

FIG. 1 shows a schematic diagram of the structure of a bone nail mounting hole positioning device according to the present invention.

### DETAILED DESCRIPTION OF THE INVENTION

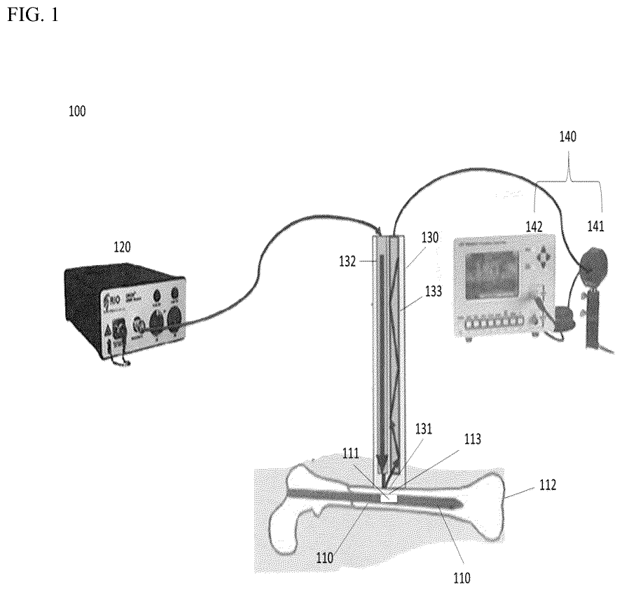

As shown in FIG. 1, a bone nail mounting hole positioning device 100 according to the invention comprises: a bone nail 110 with a screw hole 111 to be locked by a screw (not shown in the drawing), for being implanted into a body or bone of a subject 112 to be locked by the screw, in which the screw hole 111 runs through a wall 113 of the bone nail 110 for screwing therein the screw to be locked; a laser source 120 for emitting a laser light with an incident light signal; an optical fiber tube 130, including a head portion 131, a light source optical fiber 132 connected to the laser source for guiding the laser light to be incident on the bone nail 110 of the subject 112, and a receiving optical fiber 133 for receiving a reflected light signal reflected from the bone nail 110 implanted into the body or bone of the subject 112, wherein the head portion 131 of the optical fiber tube 130 is movably contacting the body or bone of the subject 112 implanted with the bone nail; and a receiving device 140, including a detector 141 connected to the receiving optical fiber 133, and a power meter 142 connected to the detector 141, wherein the incident light signal emitted by the laser source 120 is transmitted through the light source optical fiber 132 to the head portion 131 of the optical fiber tube 130, and the receiving optical fiber 133 receives the reflected light signal reflected respectively from a different location of the body or bone of the subject 112, so that the reflected light signal is transmitted to the power meter 142 for measuring via detection of the detector 141, so as to obtain and indicate an energy difference factor, in which the energy difference factor is a power ratio between the received reflected light signal and the incident light signal, and the smaller the energy difference factor, the closer to the screw hole to be locked, wherein as the incident light signal is correctly incident on the screw hole and passes therethrough, no reflected energy is generated and thus detected by the detector 141 of the receiving device 140, resulting in a larger difference between a received light energy and an emitted light energy to obtain a smaller energy difference factor indication, whereby ta correct location of the screw hole is positioned via the measurement and indication of the power meter 142.

In the bone nail mounting hole positioning device 100 of the invention, a distance between the light source optical fiber 132 as well as the receiving optical fiber 133 respectively at one end close to the body or the bone of the subject 112, and the head portion 131 of the optical fiber tube 130 is 2~5 mm. Preferably, the distance is 3 mm.

In the bone nail mounting hole positioning device 100 of the invention, the minimum value of the indication of the measured energy difference factor represents the most correct positioning.

What is claimed is:

1. A bone nail mounting hole positioning device, comprising: a bone nail with a screw hole to be locked by a screw, for being implanted into a body or bone of a subject to be locked, in which the screw hole runs through a wall of the bone nail for screwing therein the screw to be locked; a laser source for emitting a laser light with an incident light signal; an optical fiber tube, including a head portion, a light source optical fiber connected to the laser source for guiding the laser light to be incident on the bone nail of the subject, and a receiving optical fiber for receiving a reflected light signal reflected from the bone nail implanted into the body or bone of the subject, wherein the head portion of the optical fiber tube is movably contacting the body or bone of the subject implanted with the bone nail tube; and a receiving device, including a detector connected to the receiving optical fiber, and a power meter connected to the detector, wherein the incident light signal emitted by the laser source is transmitted through light source the optical fiber to the head portion of the optical fiber tube, and the receiving optical fiber receives the reflected light signal reflected respectively from a different location of the body or bone of the subject, so that the reflected light signal is transmitted to the power meter for measuring via detection of the detector, so as to obtain and indicate an energy difference factor, in which the energy difference factor is a power ratio between the received reflected light signal and the incident light signal, and the smaller the energy difference factor, the closer to the screw hole to be locked, in which the incident light signal is correctly incident on the screw hole and passes therethrough, such that no reflected energy is generated and thus detected by the detector of the receiving device, resulting in a larger difference between a received light energy and an emitted light energy to obtain a smaller energy difference factor indication, whereby a correction location of the screw hole is positioned via the measurement and indication of the power meter.

2. The bone nail mounting hole positioning device of claim 1, wherein a distance between the light source optical fiber as well as the receiving optical fiber respectively at one end close to the body or the bone of the subject, and the head portion of the movable optical fiber tube is 2~5 mm.

3. The bone nail mounting hole positioning device of claim 1, wherein a distance between the light source optical fiber as well as the receiving optical fiber at one end close to the body or bone of the subject, and the head portion of the movable optical fiber tube is 3 mm.
